(19) United States
(12) Patent Application Publication
(10) Pub. No.: US 2018/0361011 A1
(43) Pub. Date: Dec. 20, 2018

(54) CARBOXYMETHYL CHITOSAN SPONGE FORMULATION

(71) Applicant: Gyrus ACMI, Inc. d/b/a Olympus Surgical Technologies America, Southborough, MA (US)

(72) Inventor: Peter A. Norowski, Jr., Scottsdale, AZ (US)

(21) Appl. No.: 15/571,681

(22) PCT Filed: Apr. 22, 2016
(86) PCT No.: PCT/US2016/028837

§ 371 Date: Nov. 3, 2017

Related U.S. Application Data
(60) Provisional application No. 62/157,656, filed on May 6, 2015.

(51) Int. Cl. A61L 15/38; A61L 15/42; A61L 15/24; A61L 15/44; A61L 26/00

(52) U.S. Cl. CPC A61L 15/38; A61L 15/425; A61L 2400/04; A61L 15/44; A61L 26/0023; A61L 15/24

(57) ABSTRACT

Provided is a chitosan based hemostatic formulation comprising carboxymethyl chitosan, and methyl cellulose. More particularly, provided is a chitosan based hemostatic formulation comprising carboxymethyl chitosan, methyl cellulose, hydroxy ethyl cellulose, and calcium alginate. Also provided is a chitosan based hemostatic sponge comprising carboxymethyl chitosan, and methyl cellulose. More particularly, provided is a chitosan based hemostatic sponge comprising carboxymethyl chitosan, methyl cellulose, hydroxy ethyl cellulose, and calcium alginate. Further provided is a method of making and using the chitosan based hemostatic sponges.

### CROSS-REFERENCE TO RELATED PATENTS

This application claims the benefit of priority to U.S. Provisional Patent Application No. 62/157,656 filed on May 6, 2015. The entire teachings of the above application are incorporated herein by reference.

### TECHNICAL FIELD

The embodiments of this disclosure relate generally to wound dressings. In particular, the embodiments of this disclosure relate to hemostatic formulations and hemostatic sponges. Even more particularly, the embodiments of this disclosure relate to chitosan based hemostatic formulations and chitosan based hemostatic sponges and methods of making and using the chitosan based hemostatic sponges.

### BACKGROUND

U.S. Pat. No. 8,709,463 discloses compositions suitable for use in hemostatic devices. It also discloses methods of making the compositions and the hemostatic devices wherein the compositions contain biocompatible, oxidized cellulose particles having an average designated nominal particle size of about 0.035-4.35 mm and a biocompatible, water-soluble or water-swellable polysaccharide porous binder component.

U.S. Pat. No. 8,414,925 discloses an article containing N-acylchitosan manufactured by a process comprising the steps of providing a mixture containing chitosan and/or N-acylchitosan, and extruding the mixture to form an N-acylchitosan hydrogel. It also discloses an alternative process comprising the steps of providing a chitosan and/or N-acylchitosan hydrogel, and extruding the hydrogel. It further discloses an article with a memorized shape formed by fixing the N-acylchitosan hydrogel in a desired shape, and at least partially drying the fixed hydrogel. Additionally, it also discloses treating a patient by injecting the N-acylchitosan hydrogel.

U.S. Pat. No. 7,279,177 discloses a hemostatic wound dressing that utilizes a fibrous, fabric substrate made from carboxylic-oxidized cellulose. The disclosed wound dressing contains a first surface and a second surface opposing the first surface, and has a porous, polymeric matrix substantially homogeneously distributed on the first and second surfaces and through the fabric substrate, whereas the porous, polymeric matrix is made of a biocompatible, water-soluble or water-swellable cellulose polymer. The patent also discloses that the fabric substrate contains about 3 percent or more by weight of water-soluble oligosaccharides prior to distribution of the polymeric matrix on and through the fabric substrate.

U.S. Pat. No. 7,019,191 discloses methods of making wound dressings. The disclosed methods include the steps of contacting a fabric substrate having properties effective for use as a hemostat and containing fibers prepared from a biocompatible polymer, with a solution of a water-soluble or water-swellable biocompatible polymer under conditions effective to distribute the polymer solution substantially homogenously on and through the fabric substrate; transferring the fabric substrate to a lyophilization unit under conditions effective to maintain the homogeneous distribution on and throughout the substrate; and lyophilizing the fabric substrate having the polymer solution distributed on and there through.

U.S. Pat. No. 6,060,461 discloses a composition, system, articles and method for the enhancement of clotting in wounds with extravascular blood flow, especially where the surface of the tissue has been broken. The disclosed system consists of biotolerable, porous particulates applied to the surface of a wound with liquid blood thereon, wherein the porous nature of the particulate material, either free-flowing or packaged or restrained on or in a surface, enhances clotting. The patent also discloses that chemical or biochemical agents, such as additional clotting agents, therapeutic agents, antibiotics, clot strengthening agents, and the like may optionally be included on, with or within the porous particles.

US patent application publication No. 2007/0,087,061 discloses a composition, method, and use of microporous particles such as polysaccharide hemostat particle gels that activate platelet rich plasma (PRP) or other platelet-containing substances. The disclosed composition may contain microporous polysaccharaide hemostats (MPH) mixed with platelet-rich plasma, platelet-poor plasma, blood, or the like, and the disclosed method may contain mixing the MPH with platelet-rich plasma or other platelet-containing substance either by hand, in a device, or by applying the MPH directly to the wound before or after application of the platelet-containing substance. The publication also discloses that MPH can be directly applied to the bleeding wound, using the blood as a source of platelets.

### SUMMARY OF THE DISCLOSURE

In one embodiment, the disclosure provides a hemostatic formulation comprising carboxymethyl chitosan in an amount of about 45% to about 95% by weight, and methyl cellulose in an amount of about 4% to about 12% by weight.

In one embodiment, the disclosure provides a hemostatic formulation comprising carboxymethyl chitosan in an amount of about 45% to about 95% by weight, methyl cellulose in an amount of about 4% to about 12% by weight, and hydroxy ethyl cellulose in an amount of about 5% to about 15% by weight.

In one embodiment, the disclosure provides a hemostatic formulation comprising carboxymethyl chitosan in an amount of about 45% to about 95% by weight, methyl cellulose in an amount of about 4% to about 12% by weight, hydroxy ethyl cellulose in an amount of about 5% to about 15% by weight, and calcium alginate in an amount of about 10% or less by weight.

In one embodiment, the disclosure provides a hemostatic formulation comprising carboxymethyl chitosan in an amount of about 45% to about 95% by weight, methyl cellulose in an amount of about 4% to about 12% by weight, hydroxy ethyl cellulose in an amount of about 5% to about 15% by weight, calcium alginate in an amount of about 10% or less by weight, and a polyacrylate in an amount of about 10% or less by weight.

In one embodiment, the disclosure provides a hemostatic formulation consisting essentially of carboxymethyl chitosan in an amount of about 45% to about 95% by weight, methyl cellulose in an amount of about 4% to about 12% by weight, hydroxy ethyl cellulose in an amount of about 5% to about 15% by weight, and calcium alginate in an amount of about 10% or less by weight.

In one embodiment, the disclosure provides a hemostatic formulation consisting essentially of carboxymethyl chitosan in an amount of about 45% to about 95% by weight, methyl cellulose in an amount of about 4% to about 12% by weight, hydroxy ethyl cellulose in an amount of about 5% to about 15% by weight, calcium alginate in an amount of about 10% or less by weight, and a polyacrylate in an amount of about 10% or less by weight.

In one embodiment, the disclosure provides a hemostatic formulation consisting essentially of carboxymethyl chitosan in an amount of about 76% by weight, methyl cellulose in an amount of about 10% by weight, hydroxy ethyl cellulose in an amount of about 12% by weight, and calcium alginate in an amount of about 5% by weight.

In one embodiment, the disclosure provides a hemostatic sponge comprising carboxymethyl chitosan in an amount of about 45% to about 95% by weight, and methyl cellulose in an amount of about 4% to about 12% by weight.

In one embodiment, the disclosure provides a hemostatic sponge comprising carboxymethyl chitosan in an amount of about 45% to about 95% by weight, methyl cellulose in an amount of about 4% to about 12% by weight, and hydroxy ethyl cellulose in an amount of about 5% to about 15% by weight.

In one embodiment, the disclosure provides a hemostatic sponge comprising carboxymethyl chitosan in an amount of about 45% to about 95% by weight, methyl cellulose in an amount of about 4% to about 12% by weight, hydroxy ethyl cellulose in an amount of about 5% to about 15% by weight, and calcium alginate in an amount of about 10% or less by weight.

In one embodiment, the disclosure provides a hemostatic sponge comprising carboxymethyl chitosan in an amount of about 45% to about 95% by weight, methyl cellulose in an amount of about 4% to about 12% by weight, hydroxy ethyl cellulose in an amount of about 5% to about 15% by weight, calcium alginate in an amount of about 10% or less by weight, and a polyacrylate in an amount of about 10% or less by weight.

In one embodiment, the disclosure provides a hemostatic sponge consisting essentially of carboxymethyl chitosan in an amount of about 45% to about 95% by weight, methyl cellulose in an amount of about 4% to about 12% by weight, hydroxy ethyl cellulose in an amount of about 5% to about 15% by weight, and calcium alginate in an amount of about 10% or less by weight.

In one embodiment, the disclosure provides a hemostatic sponge consisting essentially of carboxymethyl chitosan in an amount of about 45% to about 95% by weight, methyl cellulose in an amount of about 4% to about 12% by weight, hydroxy ethyl cellulose in an amount of about 5% to about 15% by weight, calcium alginate in an amount of about 10% or less by weight, and a polyacrylate in an amount of about 10% or less by weight.

In one embodiment, the disclosure provides a hemostatic sponge consisting essentially of carboxymethyl chitosan in an amount of about 76% by weight, methyl cellulose in an amount of about 100/o by weight, hydroxy ethyl cellulose in an amount of about 12% by weight, and calcium alginate in an amount of about 5% by weight.

In certain embodiments, the disclosure provides a hemostatic sponge having a vertical expansion ratio of 2 or more. In certain embodiments, the disclosure provides a hemostatic sponge having a bench degradation rate of less than 20 days. In certain preferred embodiments, the hemostatic sponge has a bench degradation rate of less than 15 days. In certain embodiments, the hemostatic sponge has a vertical expansion ratio of 2 or more and a bench degradation rate of less than 20 days. In certain preferred embodiments, the hemostatic sponge has a vertical expansion ratio of 2 or more and a bench degradation rate of less than 15 days.

In certain embodiments, the disclosure provides a hemostatic sponge having a pliability of over 70 degree. In certain preferred embodiments, the hemostatic sponge has a pliability of over 90 degree. In certain preferred embodiments, the hemostatic sponge has a pliability of over 120 degree. In certain embodiments, the hemostatic sponge has a pliability of over 150 degree.

In some embodiments, the hemostatic formulation or sponge may further comprise a binding agent, a clotting accelerator, a therapeutic agent, or a combination thereof, or a mixture thereof.

The chitosan based hemostatic sponges of the present disclosure are pliable, and can conform to a bleeding site and retain good tensile and compressive strength to withstand handling during application. The chitosan based hemostatic sponges of the present disclosure can be cut into different sizes and shapes to fit the surgical needs. It can be rolled up or packed into irregular anatomic areas.

In some embodiments, the chitosan based sponges of the present disclosure may comprise a backing that is attached to at least one surface of the chitosan sponge. The backing may permit the sponge to be packaged, handled, and/or applied to a wound in a sterile and secure manner. The backing may be made of cloth, plastic, paper, film, and/or any suitable material. The backing may be attached to at least one surface of the sponge with an adhesive, stitching, staples, and/or any suitable fastener.

In another embodiment, the disclosure also provides a method of making a chitosan based wound dressing comprising dissolving the individual components in a solvent to form a solution, and freezing drying the solution to form a hemostatic sponge.

In one embodiment, the method of making a chitosan based hemostatic sponge includes providing a dry powder formulation comprising the individual components; mixing the dry formulation with purified water; lyophilizing (freeze drying) the mixture; then followed by humidification and compression, packaging and final sterilization. In one embodiment, the dry powder hemostatic formulation is mixed with purified water in an amount of about 3.5% to about 4% by weight, the mixture is subsequently lyophilized, then followed by humidification and compression, packaging and final sterilization.

In some more particular embodiments, the method of making a chitosan based sponge comprises: a) weighing and adding each component to the stirred distilled water to form a mixture, and stirring the mixture to a complete homogeneity; b) transferring the homogeneous mixture into an ultra-low freezer for a complete freezing; c) transferring the frozen mixture to a freeze dryer for freeze drying until complete drying; and d) removing the dried material from the freeze dryer (the material should be at its driest state and can be cut by razor blade to size). In some embodiments, the method of making a chitosan based sponge further or optionally includes: allowing the dried material to pick up moisture from the air until it equilibrates; compressing the moistened material (sponge) using a flat surface such as pyrex glass or PTFE sheets; and sealing and sterilizing the sponge into different shapes and sizes.

In another embodiment, the disclosure further provides a method of treating a wound/surgery/bleeding condition of a subject comprising administering a chitosan based hemostatic sponge to a wound/surgery/bleeding of a subject. In one embodiment, the disclosure provides use of a chitosan based sponge in treating a wound/surgery/bleeding condition of a subject. In certain embodiments, the chitosan based hemostatic sponges are effective in providing and maintaining hemostasis in cases of severe bleeding.

### BRIEF DESCRIPTION OF THE FIGURES/TABLES

FIG. 1 shows a comparison chart of hydration ratio and vertical expansion ratio of one prototype against PosiSep® X.

FIG. 2 shows a comparison chart of saline hydration ratio of several prototypes against PosiSep® X.

FIG. 3 shows a comparison chart of swelling ratio of several prototypes against PosiSep®.

FIG. 4 shows a comparison chart of vertical expansion ratio of several prototypes against PosiSep®.

FIG. 5 shows degradation chart of several prototypes against PosiSep® X. The units are arbitrary with 10 for no degradation and 0 meaning full degradation.

FIG. 6 shows degradation chart of additional prototypes. The units are arbitrary with 10 for no degradation and 0 meaning full degradation.

FIG. 7 shows degradation chart of additional prototypes. The units are arbitrary with 10 for no degradation and 0 meaning full degradation.

FIG. 8 shows a pliability comparison between a prototype (left) and PosiSep® X (right).

FIG. 9 shows a method of measuring a bending angle of a hemostatic sponge.

### DETAILED DESCRIPTION OF THE DISCLOSURE

### Definitions

Unless otherwise specified, the following terms and phrases shall have the meanings as set forth below:

The terms “one embodiment”, “another embodiment”, “some embodiments”, “other embodiments”, and similar expressions indicate that the embodiment or embodiments described may include a particular feature, structure, or characteristic, but every embodiment may not necessarily include the particular feature, structure, or characteristic. Moreover, such phrases are not necessarily referring to the same embodiment. Furthermore, when a particular feature, structure, or characteristic is described in connection with an embodiment, it would be within the knowledge of one skilled in the art to incorporate such feature, structure, or characteristic into other embodiments, whether or not explicitly described, unless clearly stated to the contrary. That is, the various individual elements described below, even if not explicitly shown in a particular combination, are nevertheless contemplated as being combinable with each other to form other additional embodiments or to complement and/or enrich the described embodiment or embodiments, as would be understood by one of ordinary skill in the art.

The articles “a”, “an” and “the”” are used herein to refer to one or to more than one (i.e. to at least one) of the grammatical object of the article unless otherwise clearly indicated by contrast. By way of example, “an element” means one element or more than one element.

The term “including” is used herein to mean, and is used interchangeably with, the phrase “including but not limited to”. The term “or” is used herein to mean, and is used interchangeably with, the term “and/or”, unless context clearly indicates otherwise.

The term “such as” is used herein to mean, and is used interchangeably, with the phrase “such as but not limited to”. Unless specifically stated or obvious from context, as used herein, the term “about” is understood as within a range of normal acceptance in the art, for example within standard deviations of the mean.

All numeric values are herein assumed to be modified by the term “about” whether or not explicitly indicated. The term “about”, in the context of numeric values, generally refers to a range of numbers that one of skill in the art would consider equivalent to the recited value (i.e., having the same function or result). In many instances, the term “about” may include numbers that are rounded to the nearest significant figure. Other uses of the term “about” (i.e., in a context other than numeric values) may be assumed to have their ordinary and customary definition(s), as understood from and consistent with the context of the specification, unless otherwise specified. Even more specifically, “about” can be understood as within 10%, 9%, 8%, 7%, 6%, 5%, 4%, 3%, 2%, 1%, 0.5%, 0.1%, 0.05%, or 0.01% of the stated value.

Ranges provided herein are understood to be shorthand for all of the values within the range. For example, a range of 1 to 10 is understood to include any number, combination of numbers, or sub-ranges such as 1, 1.5, 2.0, 2.8, 3.90, 4, 5, 6, 7, 8, 9, and 10.

A percent by weight of a component of a formulation or a sponge or a composition refers to a percent of a component relative to the whole weight of the formulation or the sponge or the composition. For example, a hemostatic formulation comprising carboxymethyl chitosan in an amount of about 45% to about 95% by weight refers to a formulation comprising about 45% to about 95% by weight carboxymethyl chitosan relative to the whole weight of the formulation. Or put it another way, the formulation has a content of about 45% to about 95% by weight of carboxymethyl chitosan, and other components make up the remaining about 15% to about 35% of the formulation.

As used herein, “vertical expansion ratio” refers to a ratio of the thickness along the vertical direction of a sponge when it is fully water saturated in relation to its pure dry status.

As used herein, “bench degradation rate” refers to a degradation time frame of a sponge in phosphate-buffered saline (PBS) from no degradation to full degradation. This disclosure uses an arbitrary unit of 10 for no degradation and 0 for full degradation.

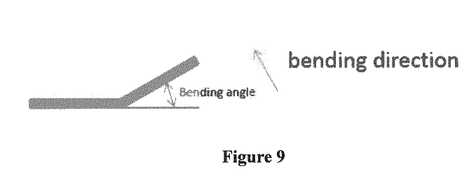

As used herein, “pliability” of a sponge is defined by a bending angle of the sponge in its dray state, whereas the bending angle is defined as the angle beyond which the sponge breaks. The bending angle is formed when one part of the sponge is fixed and the other part is pushed to bend/rotate as shown in FIG. 9. The minimum of a bending angle should be 0 degree, and the maximum of a bending angle should be 180 degree. A bending angle of 90 degree or above is considered quite pliable for a ponge.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

As used herein, the terms “treat”, “treating” or “treatment” refers, preferably, to an action to obtain a beneficial or desired clinical result including, but not limited to, alleviation or amelioration of one or more signs or symptoms of a disease or condition, diminishing the extent of disease, stability (i.e., not worsening) state of disease, amelioration or palliation of the disease state, diminishing rate of or time to progression, and remission (whether partial or total), whether detectable or undetectable. “Treatment” can also mean prolonging survival as compared to expected survival in the absence of treatment. Treatment does not need to be curative.

As used herein, “hemostatic” refers broadly to having or exhibiting the ability to significantly limit or arrest the flow of blood under the conditions referenced or apparent when the word is used. When used as a noun herein, or as the noun derivative “hemostat”, the nouns mean any substance or composition having or exhibiting the ability to significantly limit or arrest the flow of blood. The noun derivative “hemostasis” as used herein, means having blood flow in the state of significantly limited flow or arrest. The definitions herein are intended as broad descriptors and are not limited to any specific mechanism of blood coagulation or other means of blood flow limitation or arrest.

As used herein, the terms “therapeutic agent” and “therapeutic agents” refer to any agent(s) which can be used in the treatment, management, or amelioration of a disease or a condition or one or more symptoms thereof. Preferably, a therapeutic agent is an agent that is known to be useful for, or has been or is currently being used for the treatment, management, prevention, or amelioration of a disease or a condition or one or more symptoms thereof.

Chitosan (CAS registry No. 9012-76-4) is a linear polysaccharide composed of 1-(1-4)-linked D-glucosamine and N-acetyl-D-glucosamine. Chitosan is derived from chitin which is widely found in the exoskeletons of arthropods, shells of crustaceans, and the cuticles of insects. Both chitin and chitosan are promising polymers for a variety of applications. The biomedical applications of chitin and chitosan and their derivatives are of particular interest because of their biocompatibility, biodegradability and structural similarity to the glycosaminoglycans. Their medical and biomedical applications and potential applications include dressings for wound-healing, tissue engineering applications, artificial kidney membranes, drug delivery systems, absorbable sutures, hemostats, antimicrobial applications, as well as applications in dentistry, orthopedics, ophthalmology, and plastic surgery.

Chitosan is produced commercially by deacetylation of chitin. The degree of deacetylation can be determined by NMR spectroscopy, and the percentage of deacetylation in commercial chitosans ranges from 60 to 100%. On average, the molecular weight of commercially produced chitosan is between 3800 and 20,000 Daltons. A common method for the synthesis of chitosan is the deacetylation of chitin using sodium hydroxide in excess as a reagent and water as a solvent. This reaction pathway, when allowed to go to completion (complete deacetylation) yields up to 98% product. Chitosan is commercially available.

Carboxymethyl chitosan (CAS registry No. 83512-85-0) is a chitosan derivative from carboxymethylation of chitosan. The reactive sites for the carboxymethylation of chitosan are the amino and hydroxyl groups present in its chains. The choice of the appropriate reaction conditions and reagents allows the preparation of N-, O-, N,O- or N,N-carboxymethylchitosan. Thus, O-carboxymethylchitosan is predominantly obtained when the reaction is carried out at room temperature, in suspension of isopropanol/water and in the presence of monochloroacetic acid and sodium hydroxide, while this reaction yields N- and N,O-carboxymethylchitosan if it is carried out at higher temperatures. On the other hand, the N-carboxymethylchitosan may be prepared by the reaction of chitosan with glyoxylic acid followed by reduction with sodium cyanoborohydride, the degree of substitution of the derivative being determined by the reaction stoichiometry and the characteristics of the parent chitosan. The properties and applications of carboxymethylchitosan are strongly dependent on its structural characteristics, mainly the average degree of substitution and the locus, amino or hydroxyl groups, of the carboxymethylation. Generally, carboxymethyl chitosan is commercially available.

Methyl cellulose (or methylcellulose, CAS registry No. 9004-67-5) is a chemical compound derived from cellulose. It is a hydrophilic white powder in pure form and dissolves in cold (but not in hot) water, forming a clear viscous solution or gel. Like cellulose, it is not digestible, not toxic, and not an allergen. Methyl cellulose does not occur naturally and is synthetically produced by heating cellulose with caustic solution (e.g. a solution of sodium hydroxide) and treating it with methyl chloride. In the substitution reaction that follows, the hydroxyl residues (—OH functional groups) are replaced by methoxide (—OCH₃ groups).

Different kinds of methyl cellulose can be prepared depending on the number of hydroxyl groups substituted. Cellulose is a polymer consisting of numerous linked glucose molecules, each of which exposes three hydroxyl groups. The degree of substitution (DS) of a given form of methyl cellulose is defined as the average number of substituted hydroxyl groups per glucose. The theoretical maximum is thus a DS of 3.0. However more typical values are 1.3-2.6.

Methyl cellulose has a lower critical solution temperature (LCST) between 40° C. and 50° C. At temperatures below the LCST, it is readily soluble in water; above the LCST, it is not soluble, which has a paradoxical effect that heating a saturated solution of methyl cellulose will turn it solid, because methyl cellulose will precipitate out. The temperature at which this occurs depends on DS-value, with higher DS-values giving lower solubility and lower precipitation temperatures because the polar hydroxyl groups are masked.

Preparing a solution of methyl cellulose with cold water is difficult because a gel layer forms around it as the powder comes into contact with water, dramatically slowing the diffusion of water into the powder, hence the inside remains dry. A better way is to first mix the powder with hot water, so that the methyl cellulose particles are well dispersed (and so have a much higher effective surface area) in the water, and cool down this dispersion while stirring, leading to the much more rapid dissolution of those particles. Methyl cellulose is commercially available.

Hydroxy ethyl cellulose (or hydroxyethyl cellulose, CAS registry No. 9004-62-0) is a gelling and thickening agent derived from cellulose. It is widely used in cosmetics, cleaning solutions, and other household products. Hydroxyethyl cellulose is commercially available.

Calcium alginate (CAS registry No. 9005-35-0) is a water-insoluble, gelatinous, cream colored substance that can be created through the addition of aqueous calcium chloride to aqueous sodium alginate. “Alginate” is the term usually used for the salts of alginic acid, but it can also refer to all the derivatives of alginic acid and alginic acid itself; in some publications the term “aligin” is used instead of alginate. Alginate is present in the cell walls of brown algae as the calcium, magnesium and sodium salts of alginic acid. To extract the alginate, the seaweed is broken into pieces and stirred with a hot solution of an alkali, usually sodium carbonate. Over a period of about two hours, the alginate dissolves s sodium alginate to give a very thick slurry. This slurry also contains the part of the seaweed that does not dissolve, mainly cellulose. This insoluble residue must be removed from the solution. The solution is tooviscous to be filtered and must be diluted with a very large quantity of water. After dilution, the solution is forced through a filter cloth in a filter press. However, the pieces of undissolved residue are very fine and can quickly clog the filter cloth. Therefore, before filtration is started, a filter aid, such as diatomaceous earth, must be added; this holds most of the fine particles away from the surface of the filter cloth and facilitates filtration. However, filter aid is expensive and can make a significant contribution to costs. To reduce the quantity of filter aid needed, some processors force air into the extract as it is being diluted with water (the extract and diluting water are mixed in an in-line mixer into which air is forced). Fine air bubbles attach themselves to the particles of residue. The diluted extract is left standing for several hours while the air rises to the top, taking the residue particles with it. This frothy mix of air and residue is removed from the top and the solution is withdrawn from the bottom and pumped to the filter. The goal of the extraction process is to obtain dry, powdered, sodium alginate.

The calcium and magnesium salts do not dissolve in water while the sodium salt does. The rationale behind the extraction of alginate from the seaweed is to convert all the alginate salts to the sodium salt, dissolve this in water, and remove the seaweed residue by filtration. The alginate must then be recovered from the aqueous solution. The solution is very dilute and evaporation of the water is not economic. To the Sodium alginate from the initial extraction solution, a calcium salt is added. This causes calcium alginate to form with a fibrous texture; it does not dissolve in water and can be separated from it with relative ease using a metal screen. Generally, calcium alginate is commercially available.

Polyacrylic acid (PAA or Carbomer) is generic name for synthetic high molecular weight polymers of acrylic acid. They may be homopolymers of acrylic acid, crosslinked with an allyl ether pentaerythritol, allyl ether of sucrose or allyl ether of propylene. For many applications PAAs are used in the form of their alkali metal or ammonium salts. As used herein, a polyacrylate may be sodium polyacrylate, potassium polyacrylate, ammonium polyacrylate, monoethanolamine polyacrylate, diethanolamine polyacrylate, or triethanolamine polyacrylate.

As used herein, porosity is a measure of the void spaces in a material or a sponge, and is a fraction of the volume of voids over the total volume, between 0 and 1, or as a percentage between 0 and 100%. Porosity may be added to many materials by known manufacturing techniques, such as 1) co-dispersion with a differentially soluble material, and subsequent dissolution of the more soluble material, 2) particle formation from an emulsion or dispersion, with the liquid component being evaporated or otherwise removed from the solid particle after formation, 3) sintering of particles so as to leave porosity between the sintered or fused particles, 4) binding particles with a slowly soluble binder and partially removing a controlled amount of the binder, 5) providing particles with a two component, two phase system where one component is more readily removed than another solid component (as by thermal degradation, solubilization, decomposition, chemical reaction such as, chemical oxidation, aerial oxidation, chemical decomposition, etc.), and other known process for generating porosity from different or specific types of compositions and materials. There are many ways to test porosity in a substance or part, such as industrial CT scanning.

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, modifications, and equivalents as may be included within the spirit and scope of the invention as defined by the appended claims.

In one embodiment, the disclosure provides a hemostatic formulation comprising carboxymethyl chitosan in an amount of about 45% to about 95% by weight, and methyl cellulose in an amount of about 4% to about 12% by weight. In one embodiment, the formulation further comprises hydroxy ethyl cellulose. In one embodiment, the hydroxy ethyl cellulose is in an amount of about 5% to about 15% by weight. In one embodiment, the hydroxy ethyl cellulose is in an amount of about 10% to about 15% by weight. In one embodiment, the hydroxy ethyl cellulose is in an amount of about 12% by weight. In one embodiment, the formulation further comprises calcium alginate. In one embodiment, the calcium alginate is in an amount of about 10% or less by weight. In one embodiment, the calcium alginate is in an amount of about 2% to about 6% by weight. In one embodiment, the calcium alginate is in an amount of about 5% by weight. In one embodiment, the formulation further comprises a polyacrylate. In one embodiment, the polyacrylate may be sodium polyacrylate, potassium polyacrylate, ammonium polyacrylate, monoethanolamine polyacrylate, diethanolamine polyacrylate, or triethanolamine polyacrylate. In a preferred embodiment, the polyacrylate may be sodium polyacrylate. In another preferred embodiment, the sodium polyacrylate is in an amount of 10% or less by weight.

In a preferred embodiment, the disclosure provides a hemostatic formulation comprising carboxymethyl chitosan in an amount of about 70% to about 80% by weight, and methyl cellulose in an amount of about 4% to about 12% by weight. In one embodiment, the formulation further comprises hydroxy ethyl cellulose. In one embodiment, the hydroxy ethyl cellulose is in an amount of about 5% to about 15% by weight. In one embodiment, the hydroxy ethyl cellulose is in an amount of about 100/to about 15% by weight. In one embodiment, the hydroxy ethyl cellulose is in an amount of about 12% by weight. In one embodiment, the formulation further comprises calcium alginate. In one embodiment, the calcium alginate is in an amount of about 10% or less by weight. In one embodiment, the calcium alginate is in an amount of about 2% to about 6% by weight. In one embodiment, the calcium alginate is in an amount of about 5% by weight. In one embodiment, the formulation further comprises a polyacrylate. In one embodiment, the polyacrylate may be sodium polyacrylate, potassium polyacrylate, ammonium polyacrylate, monoethanolamine polyacrylate, diethanolamine polyacrylate, or triethanolamine polyacrylate. In a preferred embodiment, the polyacrylate may be sodium polyacrylate. In another preferred embodiment, the sodium polyacrylate is in an amount of 10% or less by weight.

In another preferred embodiment, the disclosure provides a hemostatic formulation comprising carboxymethyl chitosan in an amount of about 70% to about 80% by weight, and methyl cellulose in an amount of about 5% to about 10% by weight. In one embodiment, the formulation further comprises hydroxy ethyl cellulose. In one embodiment, the hydroxy ethyl cellulose is in an amount of about 5% to about 15% by weight. In one embodiment, the hydroxy ethyl cellulose is in an amount of about 10% to about 15% by weight. In one embodiment, the hydroxy ethyl cellulose is in an amount of about 12% by weight. In one embodiment, the formulation further comprises calcium alginate. In one embodiment, the calcium alginate is in an amount of about 10% or less by weight. In one embodiment, the calcium alginate is in an amount of about 2% to about 6% by weight. In one embodiment, the calcium alginate is in an amount of about 5% by weight. In one embodiment, the formulation further comprises a polyacrylate. In one embodiment, the polyacrylate may be sodium polyacrylate, potassium polyacrylate, ammonium polyacrylate, monoethanolamine polyacrylate, diethanolamine polyacrylate, or triethanolamine polyacrylate. In a preferred embodiment, the polyacrylate may be sodium polyacrylate. In another preferred embodiment, the sodium polyacrylate is in an amount of 10% or less by weight.

In one embodiment, the disclosure provides a hemostatic formulation comprising carboxymethyl chitosan in an amount of about 45% to about 95% by weight, methyl cellulose in an amount of about 4% to about 12% by weight, hydroxy ethyl cellulose in an amount of about 5% to about 15% by weight, and calcium alginate in an amount of about 10% or less by weight.

In a preferred embodiment, the disclosure provides a hemostatic formulation comprising carboxymethyl chitosan in an amount of about 70% to about 80% by weight, methyl cellulose in an amount of about 4% to about 12% by weight, hydroxy ethyl cellulose in an amount of about 5% to about 15% by weight, and calcium alginate in an amount of about 10% or less by weight.

In a preferred embodiment, the disclosure provides a hemostatic formulation comprising carboxymethyl chitosan in an amount of about 70% to about 80% by weight, methyl cellulose in an amount of about 5% to about 10% by weight, hydroxy ethyl cellulose in an amount of about 5% to about 15% by weight, and calcium alginate in an amount of about 10% or less by weight.

In a preferred embodiment, the disclosure provides a hemostatic formulation comprising carboxymethyl chitosan in an amount of about 70% to about 80% by weight, methyl cellulose in an amount of about 5% to about 10% by weight, hydroxy ethyl cellulose in an amount of about 10% to about 15% by weight, and calcium alginate in an amount of about 10% or less by weight.

In a preferred embodiment, the disclosure provides a hemostatic formulation comprising carboxymethyl chitosan in an amount of about 70% to about 80% by weight, methyl cellulose in an amount of about 5% to about 10% by weight, hydroxy ethyl cellulose in an amount of about 10% to about 15% by weight, and calcium alginate in an amount of about 2% to about 6% by weight.

In one embodiment, the disclosure provides a hemostatic formulation consisting essentially of carboxymethyl chitosan in an amount of about 45% to about 95% by weight, methyl cellulose in an amount of about 4% to about 12% by weight, hydroxy ethyl cellulose in an amount of about 5% to about 15% by weight, and calcium alginate in an amount of about 10% or less by weight.

In a preferred embodiment, the disclosure provides a hemostatic formulation consisting essentially of carboxymethyl chitosan an amount of about 70% to about 80% by weight, methyl cellulose in an amount of about 4% to about 12% by weight, hydroxy ethyl cellulose in an amount of about 5% to about 15% by weight, and calcium alginate in an amount of about 10% or less by weight.

In one embodiment, the disclosure provides a hemostatic formulation comprising carboxymethyl chitosan in an amount of about 45% to about 95% by weight, methyl cellulose in an amount of about 4% to about 12% by weight, hydroxy ethyl cellulose in an amount of about 5% to about 15% by weight, calcium alginate in an amount of about 10% or less by weight, and sodium polyacrylate is in an amount of 10% or less by weight.

In a preferred embodiment, the disclosure provides a hemostatic formulation consisting essentially of carboxymethyl chitosan in an amount of about 70% to about 80% by weight, methyl cellulose in an amount of about 5% to about 10% by weight, hydroxy ethyl cellulose in an amount of about 5% to about 15% by weight, and calcium alginate in an amount of about 10% or less by weight.

In a preferred embodiment, the disclosure provides a hemostatic formulation consisting essentially of carboxymethyl chitosan in an amount of about 70% to about 80% by weight, methyl cellulose in an amount of about 5% to about 10% by weight, hydroxy ethyl cellulose in an amount of about 10% to about 15% by weight, and calcium alginate in an amount of about 10% or less by weight.

In a preferred embodiment, the disclosure provides a hemostatic formulation consisting essentially of carboxymethyl chitosan in an amount of about 70% to about 80% by weight, methyl cellulose in an amount of about 5% to about 10% by weight, hydroxy ethyl cellulose in an amount of about 10% to about 15% by weight, and calcium alginate in an amount of about 2% to about 6% by weight.

In one embodiment, the disclosure provides a hemostatic formulation consisting essentially of carboxymethyl chitosan in an amount of about 76% by weight, methyl cellulose in an amount of about 10% by weight, hydroxy ethyl cellulose in an amount of about 12% by weight, and calcium alginate in an amount of about 5% by weight.

In one embodiment, the disclosure provides a hemostatic sponge comprising carboxymethyl chitosan in an amount of about 45% to about 95% by weight, and methyl cellulose in an amount of about 4% to about 12% by weight. In one embodiment, the sponge further comprises hydroxy ethyl cellulose. In one embodiment, the hydroxy ethyl cellulose is in an amount of about 5% to about 15% by weight. In one embodiment, the hydroxy ethyl cellulose is in an amount of about 10% to about 15% by weight. In one embodiment, the hydroxy ethyl cellulose is in an amount of about 12% by weight. In one embodiment, the sponge further comprises calcium alginate. In one embodiment, the calcium alginate is in an amount of about 10% or less by weight. In one embodiment, the calcium alginate is in an amount of about 2% to about 6% by weight. In one embodiment, the calcium alginate is in an amount of about 5% by weight. In one embodiment, the sponge further comprises a polyacrylate. In one embodiment, the polyacrylate may be sodium polyacrylate, potassium polyacrylate, ammonium polyacrylate, monoethanolamine polyacrylate, diethanolamine polyacrylate, or triethanolamine polyacrylate. In a preferred embodiment, the polyacrylate may be sodium polyacrylate. In another preferred embodiment, the sodium polyacrylate is in an amount of 10% or less by weight. In certain embodiments, the hemostatic sponge has a vertical expansion ratio of 2 or more. In certain embodiments, the hemostatic sponge has a bench degradation rate of less than 20 days. In certain preferred embodiments, the hemostatic sponge has a bench degradation rate of less than 15 days. In certain embodiments, the hemostatic sponge has a vertical expansion ratio of 2 or more and a bench degradation rate of less than 20 days. In certain preferred embodiments, the hemostatic sponge has a vertical expansion ratio of 2 or more and a bench degradation rate of less than 15 days. In certain embodiments, the hemostatic sponge has a pliability of over 70 degree. In certain preferred embodiments, the hemostatic sponge has a pliability of over 90 degree. In certain preferred embodiments, the hemostatic sponge has a pliability of over 120 degree. In certain embodiments, the hemostatic sponge has a pliability of over 150 degree.

In a preferred embodiment, the disclosure provides a hemostatic sponge comprising carboxymethyl chitosan in an amount of about 70% to about 80% by weight, and methyl cellulose in an amount of about 4% to about 12% by weight. In one embodiment, the sponge further comprises hydroxy ethyl cellulose. In one embodiment, the hydroxy ethyl cellulose is in an amount of about 5% to about 15% by weight. In one embodiment, the hydroxy ethyl cellulose is in an amount of about 10% to about 15% by weight. In one embodiment, the hydroxy ethyl cellulose is in an amount of about 12% by weight. In one embodiment, the sponge further comprises calcium alginate. In one embodiment, the calcium alginate is in an amount of about 10% or less by weight. In one embodiment, the calcium alginate is in an amount of about 2% to about 6% by weight. In one embodiment, the calcium alginate is in an amount of about 5% by weight. In one embodiment, the hemostatic sponge further comprises sodium polyacrylate. In one embodiment, the sodium polyacrylate is in an amount of 10% or less by weight. In certain embodiments, the hemostatic sponge has a vertical expansion ratio of 2 or more. In certain embodiments, the hemostatic sponge has a bench degradation rate of less than 20 days. In certain preferred embodiments, the hemostatic sponge has a bench degradation rate of less than 15 days. In certain embodiments, the hemostatic sponge has a vertical expansion ratio of 2 or more and a bench degradation rate of less than 20 days. In certain preferred embodiments, the hemostatic sponge has a vertical expansion ratio of 2 or more and a bench degradation rate of less than 15 days. In certain embodiments, the hemostatic sponge has a pliability of over 70 degree. In certain preferred embodiments, the hemostatic sponge has a pliability of over 90 degree. In certain preferred embodiments, the hemostatic sponge has a pliability of over 120 degree. In certain embodiments, the hemostatic sponge has a pliability of over 150 degree.

In another preferred embodiment, the disclosure provides a hemostatic sponge comprising carboxymethyl chitosan in an amount of about 70% to about 80% by weight, and methyl cellulose in an amount of about 5% to about 10% by weight. In one embodiment, the sponge further comprises hydroxy ethyl cellulose. In one embodiment, the hydroxy ethyl cellulose is in an amount of about 5% to about 15% by weight. In one embodiment, the hydroxy ethyl cellulose is in an amount of about 10% to about 15% by weight. In one embodiment, the hydroxy ethyl cellulose is in an amount of about 12% by weight. In one embodiment, the sponge further comprises calcium alginate. In one embodiment, the calcium alginate is in an amount of about 10% or less by weight. In one embodiment, the calcium alginate is in an amount of about 2% to about 6% by weight. In one embodiment, the calcium alginate is in an amount of about 5% by weight. In one embodiment, the hemostatic sponge further comprises sodium polyacrylate. In one embodiment, the sodium polyacrylate is in an amount of 10% or less by weight. In certain embodiments, the hemostatic sponge has a vertical expansion ratio of 2 or more. In certain embodiments, the hemostatic sponge has a bench degradation rate of less than 20 days. In certain preferred embodiments, the hemostatic sponge has a bench degradation rate of less than 15 days. In certain embodiments, the hemostatic sponge has a vertical expansion ratio of 2 or more and a bench degradation rate of less than 20 days. In certain preferred embodiments, the hemostatic sponge has a vertical expansion ratio of 2 or more and a bench degradation rate of less than 15 days. In certain embodiments, the hemostatic sponge has a pliability of over 70 degree. In certain preferred embodiments, the hemostatic sponge has a pliability of over 90 degree. In certain preferred embodiments, the hemostatic sponge has a pliability of over 120 degree. In certain embodiments, the hemostatic sponge has a pliability of over 150 degree.

In one embodiment, the disclosure provides a hemostatic sponge comprising carboxymethyl chitosan in an amount of about 45% to about 95% by weight, methyl cellulose in an amount of about 4% to about 12% by weight, hydroxy ethyl cellulose in an amount of about 5% to about 15% by weight, and calcium alginate an amount of about 10% or less by weight. In certain embodiments, the hemostatic sponge has a vertical expansion ratio of 2 or more. In certain embodiments, the hemostatic sponge has a bench degradation rate of less than 20 days. In certain preferred embodiments, the hemostatic sponge has a bench degradation rate of less than 15 days. In certain embodiments, the hemostatic sponge has a vertical expansion ratio of 2 or more and a bench degradation rate of less than 20 days. In certain preferred embodiments, the hemostatic sponge has a vertical expansion ratio of 2 or more and a bench degradation rate of less than 15 days. In certain embodiments, the hemostatic sponge has a pliability of over 70 degree. In certain preferred embodiments, the hemostatic sponge has a pliability of over 90 degree. In certain preferred embodiments, the hemostatic sponge has a pliability of over 120 degree. In certain embodiments, the hemostatic sponge has a pliability of over 150 degree.

In a preferred embodiment, the disclosure provides a hemostatic sponge comprising carboxymethyl chitosan in an amount of about 70% to about 80% by weight, methyl cellulose in an amount of about 4% to about 12% by weight, hydroxy ethyl cellulose in an amount of about 5% to about 15% by weight, and calcium alginate in an amount of about 10% or less by weight. In certain embodiments, the hemostatic sponge has a vertical expansion ratio of 2 or more. In certain embodiments, the hemostatic sponge has a bench degradation rate of less than 20 days. In certain preferred embodiments, the hemostatic sponge has a bench degradation rate of less than 15 days. In certain embodiments, the hemostatic sponge has a vertical expansion ratio of 2 or more and a bench degradation rate of less than 20 days. In certain preferred embodiments, the hemostatic sponge has a vertical expansion ratio of 2 or more and a bench degradation rate of less than 15 days. In certain embodiments, the hemostatic sponge has a pliability of over 70 degree. In certain preferred embodiments, the hemostatic sponge has a pliability of over 90 degree. In certain preferred embodiments, the hemostatic sponge has a pliability of over 120 degree. In certain embodiments, the hemostatic sponge has a pliability of over 150 degree.

In a preferred embodiment, the disclosure provides a hemostatic sponge comprising carboxymethyl chitosan in an amount of about 70% to about 80% by weight, methyl cellulose in an amount of about 5% to about 10% by weight, hydroxy ethyl cellulose in an amount of about 5% to about 15% by weight, and calcium alginate in an amount of about 10% or less by weight. In certain embodiments, the hemostatic sponge has a vertical expansion ratio of 2 or more. In certain embodiments, the hemostatic sponge has a bench degradation rate of less than 20 days. In certain preferred embodiments, the hemostatic sponge has a bench degradation rate of less than 15 days. In certain embodiments, the hemostatic sponge has a vertical expansion ratio of 2 or more and a bench degradation rate of less than 20 days. In certain preferred embodiments, the hemostatic sponge has a vertical expansion ratio of 2 or more and a bench degradation rate of less than 15 days. In certain embodiments, the hemostatic sponge has a pliability of over 70 degree. In certain preferred embodiments, the hemostatic sponge has a pliability of over 90 degree. In certain preferred embodiments, the hemostatic sponge has a pliability of over 120 degree. In certain embodiments, the hemostatic sponge has a pliability of over 150 degree.

In a preferred embodiment, the disclosure provides a hemostatic sponge comprising carboxymethyl chitosan in an amount of about 70% to about 80% by weight, methyl cellulose in an amount of about 5% to about 10% by weight, hydroxy ethyl cellulose in an amount of about 10% to about 15% by weight, and calcium alginate in an amount of about 10% or less by weight. In certain embodiments, the hemostatic sponge has a vertical expansion ratio of 2 or more. In certain embodiments, the hemostatic sponge has a bench degradation rate of less than 20 days. In certain preferred embodiments, the hemostatic sponge has a bench degradation rate of less than 15 days. In certain embodiments, the hemostatic sponge has a vertical expansion ratio of 2 or more and a bench degradation rate of less than 20 days. In certain preferred embodiments, the hemostatic sponge has a vertical expansion ratio of 2 or more and a bench degradation rate of less than 15 days. In certain embodiments, the hemostatic sponge has a pliability of over 70 degree. In certain preferred embodiments, the hemostatic sponge has a pliability of over 90 degree. In certain preferred embodiments, the hemostatic sponge has a pliability of over 120 degree. In certain embodiments, the hemostatic sponge has a pliability of over 150 degree.

In a preferred embodiment, the disclosure provides a hemostatic sponge comprising carboxymethyl chitosan in an amount of about 70% to about 80% by weight, methyl cellulose in an amount of about 5% to about 10% by weight, hydroxy ethyl cellulose in an amount of about 10% to about 15% by weight, and calcium alginate in an amount of about 2% to about 6% by weight. In certain embodiments, the hemostatic sponge has a vertical expansion ratio of 2 or more. In certain embodiments, the hemostatic sponge has a bench degradation rate of less than 20 days. In certain preferred embodiments, the hemostatic sponge has a bench degradation rate of less than 15 days. In certain embodiments, the hemostatic sponge has a vertical expansion ratio of 2 or more and a bench degradation rate of less than 20 days. In certain preferred embodiments, the hemostatic sponge has a vertical expansion ratio of 2 or more and a bench degradation rate of less than 15 days. In certain embodiments, the hemostatic sponge has a pliability of over 70 degree. In certain preferred embodiments, the hemostatic sponge has a pliability of over 90 degree. In certain preferred embodiments, the hemostatic sponge has a pliability of over 120 degree. In certain embodiments, the hemostatic sponge has a pliability of over 150 degree.

In one embodiment, the disclosure provides a hemostatic sponge comprising carboxymethyl chitosan in an amount of about 45% to about 95% by weight, methyl cellulose in an amount of about 4% to about 12% by weight, hydroxy ethyl cellulose in an amount of about 5% to about 15% by weight, calcium alginate an amount of about 10% or less by weight, and sodium polyacrylate is in an amount of 10% or less by weight. In certain embodiments, the hemostatic sponge has a vertical expansion ratio of 2 or more. In certain embodiments, the hemostatic sponge has a bench degradation rate of less than 20 days. In certain preferred embodiments, the hemostatic sponge has a bench degradation rate of less than 15 days. In certain embodiments, the hemostatic sponge has a vertical expansion ratio of 2 or more and a bench degradation rate of less than 20 days. In certain preferred embodiments, the hemostatic sponge has a vertical expansion ratio of 2 or more and a bench degradation rate of less than 15 days. In certain embodiments, the hemostatic sponge has a pliability of over 70 degree. In certain preferred embodiments, the hemostatic sponge has a pliability of over 90 degree. In certain preferred embodiments, the hemostatic sponge has a pliability of over 120 degree. In certain embodiments, the hemostatic sponge has a pliability of over 150 degree.

In one embodiment, the disclosure provides a hemostatic sponge consisting essentially of carboxymethyl chitosan in an amount of about 45% to about 95% by weight, methyl cellulose in an amount of about 4% to about 12% by weight, hydroxy ethyl cellulose in an amount of about 5% to about 15% by weight, and calcium alginate in an amount of about 10% or less by weight. In certain embodiments, the hemostatic sponge has a vertical expansion ratio of 2 or more. In certain embodiments, the hemostatic sponge has a bench degradation rate of less than 20 days. In certain preferred embodiments, the hemostatic sponge has a bench degradation rate of less than 15 days. In certain embodiments, the hemostatic sponge has a vertical expansion ratio of 2 or more and a bench degradation rate of less than 20 days. In certain preferred embodiments, the hemostatic sponge has a vertical expansion ratio of 2 or more and a bench degradation rate of less than 15 days. In certain embodiments, the hemostatic sponge has a pliability of over 70 degree. In certain preferred embodiments, the hemostatic sponge has a pliability of over 90 degree. In certain preferred embodiments, the hemostatic sponge has a pliability of over 120 degree. In certain embodiments, the hemostatic sponge has a pliability of over 150 degree.

In a preferred embodiment, the disclosure provides a hemostatic sponge consisting essentially of carboxymethyl chitosan in an amount of about 70% to about 80% by weight, methyl cellulose in an amount of about 4% to about 12% by weight, hydroxy ethyl cellulose in an amount of about 5% to about 15% by weight, and calcium alginate in an amount of about 10% or less by weight. In certain embodiments, the hemostatic sponge has a vertical expansion ratio of 2 or more. In certain embodiments, the hemostatic sponge has a bench degradation rate of less than 20 days. In certain preferred embodiments, the hemostatic sponge has a bench degradation rate of less than 15 days. In certain embodiments, the hemostatic sponge has a vertical expansion ratio of 2 or more and a bench degradation rate of less than 20 days. In certain preferred embodiments, the hemostatic sponge has a vertical expansion ratio of 2 or more and a bench degradation rate of less than 15 days. In certain embodiments, the hemostatic sponge has a pliability of over 70 degree. In certain preferred embodiments, the hemostatic sponge has a pliability of over 90 degree. In certain preferred embodiments, the hemostatic sponge has a pliability of over 120 degree. In certain embodiments, the hemostatic sponge has a pliability of over 150 degree.

In a preferred embodiment, the disclosure provides a hemostatic sponge consisting essentially of carboxymethyl chitosan in an amount of about 70% to about 80% by weight, methyl cellulose in an amount of about 5% to about 10% by weight, hydroxy ethyl cellulose in an amount of about 5% to about 15% by weight, and calcium alginate in an amount of about 10% or less by weight. In certain embodiments, the hemostatic sponge has a vertical expansion ratio of 2 or more. In certain embodiments, the hemostatic sponge has a bench degradation rate of less than 20 days. In certain preferred embodiments, the hemostatic sponge has a bench degradation rate of less than 15 days. In certain embodiments, the hemostatic sponge has a vertical expansion ratio of 2 or more and a bench degradation rate of less than 20 days. In certain preferred embodiments, the hemostatic sponge has a vertical expansion ratio of 2 or more and a bench degradation rate of less than 15 days. In certain embodiments, the hemostatic sponge has a pliability of over 70 degree. In certain preferred embodiments, the hemostatic sponge has a pliability of over 90 degree. In certain preferred embodiments, the hemostatic sponge has a pliability of over 120 degree. In certain embodiments, the hemostatic sponge has a pliability of over 150 degree.

In a preferred embodiment, the disclosure provides a hemostatic sponge consisting essentially of carboxymethyl chitosan in an amount of about 70% to about 80% by weight, methyl cellulose in an amount of about 5% to about 10% by weight, hydroxy ethyl cellulose in an amount of about 10% to about 15% by weight, and calcium alginate in an amount of about 10% or less by weight. In certain embodiments, the hemostatic sponge has a vertical expansion ratio of 2 or more. In certain embodiments, the hemostatic sponge has a bench degradation rate of less than 20 days. In certain preferred embodiments, the hemostatic sponge has a bench degradation rate of less than 15 days. In certain embodiments, the hemostatic sponge has a vertical expansion ratio of 2 or more and a bench degradation rate of less than 20 days. In certain preferred embodiments, the hemostatic sponge has a vertical expansion ratio of 2 or more and a bench degradation rate of less than 15 days. In certain embodiments, the hemostatic sponge has a pliability of over 70 degree. In certain preferred embodiments, the hemostatic sponge has a pliability of over 90 degree. In certain preferred embodiments, the hemostatic sponge has a pliability of over 120 degree. In certain embodiments, the hemostatic sponge has a pliability of over 150 degree.

In a preferred embodiment, the disclosure provides a hemostatic sponge consisting essentially of carboxymethyl chitosan in an amount of about 70% to about 80% by weight, methyl cellulose in an amount of about 5% to about 10% by weight, hydroxy ethyl cellulose in an amount of about 10% to about 15%, and calcium alginate in an amount of about 2% to about 6% by weight. In certain embodiments, the hemostatic sponge has a vertical expansion ratio of 2 or more. In certain embodiments, the hemostatic sponge has a bench degradation rate of less than 20 days. In certain preferred embodiments, the hemostatic sponge has a bench degradation rate of less than 15 days. In certain embodiments, the hemostatic sponge has a vertical expansion ratio of 2 or more and a bench degradation rate of less than 20 days. In certain preferred embodiments, the hemostatic sponge has a vertical expansion ratio of 2 or more and a bench degradation rate of less than 15 days. In certain embodiments, the hemostatic sponge has a pliability of over 70 degree. In certain preferred embodiments, the hemostatic sponge has a pliability of over 90 degree. In certain preferred embodiments, the hemostatic sponge has a pliability of over 120 degree. In certain embodiments, the hemostatic sponge has a pliability of over 150 degree.

In one embodiment, the disclosure provides a hemostatic sponge consisting essentially of carboxymethyl chitosan in an amount of about 76% by weight, methyl cellulose in an amount of about 10% by weight, hydroxy ethyl cellulose in an amount of about 12% by weight, and calcium alginate in an amount of about 5% by weight. In certain embodiments, the hemostatic sponge has a vertical expansion ratio of 2 or more. In certain embodiments, the hemostatic sponge has a bench degradation rate of less than 20 days. In certain preferred embodiments, the hemostatic sponge has a bench degradation rate of less than 15 days. In certain embodiments, the hemostatic sponge has a vertical expansion ratio of 2 or more and a bench degradation rate of less than 20 days. In certain preferred embodiments, the hemostatic sponge has a vertical expansion ratio of 2 or more and a bench degradation rate of less than 15 days. In certain embodiments, the hemostatic sponge has a pliability of over 70 degree. In certain preferred embodiments, the hemostatic sponge has a pliability of over 90 degree. In certain preferred embodiments, the hemostatic sponge has a pliability of over 120 degree. In certain embodiments, the hemostatic sponge has a pliability of over 150 degree.

In certain embodiments, the disclosure provides a hemostatic sponge having a porosity of at least 20%. In certain embodiments, the disclosure provides a hemostatic sponge having a porosity of at least 30%. In certain embodiments, the disclosure provides a hemostatic sponge having a porosity of at least 40%.

In some embodiments, the hemostatic formulation or sponge may further comprise a binding agent, a clotting accelerator, a therapeutic agent, or a combination thereof, or a mixture thereof. A binding agent may be dissolved with the individual components in a solvent. A binding agent may further increase or decrease the flexibility of sponge, the liquid holding capacity of sponge, and/or the rate at which sponge absorbs liquid. Examples of binding agents include polyethylene glycol, glycerol, sorbitol, erythritol, propylene glycol, pentaerythritol, glycerol esters, hydroxypropylmethyl cellulose (HPMC), hydroxypropylcellulose (HPC), hydroxypropylethylcellulose (HPEC), xanthum gum, guar gum, gum Arabic, and sodium carboxylmethylcellulose (CMC). Binding agents may be soluble in water and/or other solvents. In some embodiments, the hemostatic formulation or sponge may comprise a single binding agent or a combination of different binding agents. In some embodiments, the hemostatic formulation or sponge may not comprise any binding agents. In such embodiments, the individual components may adhere together without a binding agent.

In some embodiments, the hemostatic formulation or sponge may comprise a clotting accelerator to speed the clotting process. A clotting accelerator may be dissolved with the individual components in a solvent. The clotting accelerator may be calcium chloride, prothrombin, vitamin K, fibrin, fibrinogen, and/or any suitable clotting accelerator. The amount of clotting accelerator added to the sponge formulation may depend upon the application but it may be a smaller percentage by weight or a larger percentage by weight as compared to the individual components of the formulation or the sponge. The hemostatic formulation or sponge may comprise a single clotting accelerator or a combination of different clotting accelerators. In some embodiments, such as where the individual components are sufficient to clot blood by itself, the hemostatic formulation or sponge may not comprise any clotting accelerators.

In some embodiments, the hemostatic formulation or sponge may further comprise one or more therapeutic agents. The one or more therapeutic agents may include anti-inflammatory agents, antibiotics, antiviral agents, antifungals, antiprotozoal agents, immunosuppressive agents, other suitable drugs, or combinations thereof, or mixtures thereof. The one or more therapeutic agents may be mixed with the hemostatic sponge formulation while the sponge is being made or may be applied to a surface of the sponge after manufacture.

Anti-inflammatory agents as used herein may be glucocorticosteroids or non-steroidal anti-inflammatory drugs (“NSAIDs”).

Glucocorticosteroids may be 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, a physiologically acceptable salt thereof, a derivative thereof, a combination thereof, or a mixture thereof.

NSAIDs may be aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cimnetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (e.g., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide O-acetic acid, salicylsulfuric acid, salsalate, sulfasalazine), thiazinecarboxamides (e.g., ampiroxicam, droxicam, isoxicam, lornoxicam, piroxicam, tenoxicam), ε-acetamidocaproic acid, S-(5′-adenosyl)-L-methionine, 3-amino-4-hydroxybutyric acid, amixetrine, bendazac, benzydamine, α-bisabolol, bucolome, difenpiramide, ditazol, emorfazone, fepradinol, guaiazalene, nabumetone, nimesulide, oxaceprol, paranyline, perisoxal, proquazone, superoxide dismutase, tenidap, zileuton, a physiologically acceptable salt thereof, a combination thereof, or a mixture thereof.

Antibiotics may be doxorubicin, aminoglycosides (e.g., amikacin, apramycin, arbekacin, bambermycins, butirosin, dibekacin, dihydrostreptomycin, fortimicin(s), gentamicin, isepamicin, kanamycin, micronomicin, neomycin, neomycin undecylenate, netilmicin, paromomycin, ribostamycin, sisomicin, spectinomycin, streptomycin, tobramycin, trospectomycin), amphenicols (e.g., azidamfenicol, chloramphenicol, florfenicol, thiamphenicol), ansamycins (e.g., rifamide, rifampin, rifamycin SV, rifapentine, rifaximin), β-lactams (e.g., carbacephems (e.g., loracarbet)), carbapenems (e.g., biapenem, imipenem, meropenem, panipenem), cephalosporins (e.g., cefaclor, cefadroxil, cefamandole, cefatrizine, cefazedone, cefazolin, cefcapene pivoxil, cefclidin, cefdinir, cefditoren, cefepime, cefetamet, cefixime, cefinenoxime, cefodizime, cefonicid, cefoperazone, ceforamide, cefotaxime, cefotiam, cefozopran, cefpimizole, cefpiramide, cefpirome, cefpodoxime proxetil, cefprozil, cefroxadine, cefsulodin, ceftazidime, cefteram, ceftezole, ceftibuten, ceftizoxime, cefiriaxone, cefuroxime, cefuzonam, cephacetrile sodium, cephalexin, cephaloglycin, cephaloridine, cephalosporin, cephalothin, cephapirin sodium, cephradine, pivcefalexin), cephamycins (e.g., cefbuperazone, cefinetazole, cefininox, cefotetan, cefoxitin), monobactams (e.g., aztreonam, carumonam, tigemonam), oxacephems, flomoxef, moxalactam), penicillins (e.g., amdinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin G benethamine, penicillin G benzathine, penicillin G benzhydrylamine, penicillin G calcium, penicillin G hydrabamine, penicillin G potassium, penicillin G procaine, penicillin N, penicillin O, penicillin V, penicillin V benzathine, penicillin V hydrabamine, penimepicycline, phenethicillin potassium, piperacillin, pivampicillin, propicillin, quinacillin, sulbenicillin, sultamicillin, talampicillin, temocillin, ticarcillin), lincosamides (e.g., clindamycin, lincomycin), macrolides (e.g., azithromycin, carbomycin, clarithromycin, dirithromycin, erythromycin, erythromycin acistrate, erythromycin estolate, erythromycin glucoheptonate, erythromycin lactobionate, erythromycin propionate, erythromycin stearate, josamycin, leucomycins, midecamycins, miokamycin, oleandomycin, primycin, rokitamycin, rosaramicin, roxithromycin, spiramycin, troleandomycin), polypeptides (e.g., amphomycin, bacitracin, capreomycin, colistin, enduracidin, enviomycin, fusafungine, gramicidin S, gramicidin(s), mikamycin, polymyxin, pristinamycin, ristocetin, teicoplanin, thiostrepton, tuberactinomycin, tyrocidine, tyrothricin, vancomycin, viomycin, virginiamycin, zinc bacitracin), tetracyclines (e.g., apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, tetracycline), or others (e.g., cycloserine, mupirocin, tuberin).

Additional antibiotics may be the synthetic antibiotics such as 2,4-diaminopyrimidines (e.g., brodimoprim, tetroxoprim, trimethoprim), nitrofurans (e.g., furaltadone, furazolium chloride, nifuradene, nifuratel, nifurfoline, nifurpirinol, nifurprazine, nifurtoinol, nitrofurantoin), quinolones and analogs (e.g., cinoxacin, ciprofloxacin, clinafloxacin, difloxacin, enoxacin, fleroxacin, flumequine, grepafloxacin, lomefloxacin, miloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, trovafloxacin), sulfonamides (e.g., acetyl sulfamethoxypyrazine, benzylsulfamide, chloramine-B, chloramine-T, dichloramine T, n²-formylsulfisomidine, n⁴-β-D-glucosylsulfanilamide, mafenide, 4′-(methylsulfamoyl)sulfanilanilide, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidochrysoidine, sulfamoxole, sulfanilamide, 4-sulfanilamidosalicylic acid, n⁴-sulfanilylsulfanilamide, sulfanilylurea, n-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine, sulfisoxazole) sulfones (e.g., acedapsone, acediasulfone, acetosulfone sodium, dapsone, diathymosulfone, glucosulfone sodium, solasulfone, succisulfone, sulfanilic acid, p-sulfanilylbenzylamine, sulfoxone sodium, thiazolsulfone), and others (e.g., clofoctol, hexedine, methenamine, methenamine anhydromethylene citrate, methenamine hippurate, methenamine mandelate, methenamine sulfosalicylate, nitroxoline, taurolidine, xibomol).

Immunosuppressive agents may be dexamethasone, cyclosporin A, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, trimetrexate), purine analogs (e.g., cladribine, fludarabine, 6-mercaptopurine, thiamiprine, thiaguanine), pyrimidine analogs (e.g., ancitabine, azacitidine, 6-azauridine, carmofur, cytarabine, doxifluridine, emitefir, enocitabine, floxuridine, fluorouracil, gemcitabine, tegafur), fluocinolone, triaminolone, anecortave acetate, fluorometholone, medrysone, or prednisolone.

Antifungal agents may be polyenes (e.g., amphotericin B, candicidin, dermostatin, filipin, fungichromin, hachimycin, hamycin, lucensomycin, mepartricin, natamycin, nystatin, pecilocin, perimycin), azaserine, griseofilvin, oligomycins, neomycin undecylenate, pyiroInitrin, siccanin, tubercidin, viridin, allylamines (e.g., butenafine, naftifine, terbinafine), imidazoles (e.g., bifonazole, butoconazole, chlordantoin, chlormidazole, cloconazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole), thiocarbamates (e.g., tolciclate, tolindate, tolnaftate), triazoles (e.g., fluconazole, itraconazole, saperconazole, terconazole), acrisorcin, amorolfine, biphenamine, bromosalicylchloranilide, buclosamide, calcium propionate, chlorphenesin, ciclopirox, cloxyquin, coparaffinate, diamthazole dihydrochloride, exalamide, flucytosine, halethazole, hexetidine, loflucarban, nifuratel, potassium iodide, propionic acid, pyrithione, salicylanilide, sodium propionate, sulbentine, tenonitrozole, triacetin, ujothion, undecylenic acid, or zinc propionate.

Antiviral agents may be acyclovir, carbovir, famciclovir, ganciclovir, penciclovir, or zidovudine.

Antiprotozoal agents may be pentamidine isethionate, quinine, chloroquine, or mefloquine

The chitosan based hemostatic sponges of the present disclosure are pliable, and can conform to a bleeding site and retain good tensile and compressive strength to withstand handling during application. The chitosan sponges can be cut into different sizes and shapes to fit the surgical needs. It can be rolled up or packed into irregular anatomic areas.

In some embodiments, the chitosan sponges may comprise a backing that is attached to at least one surface of the sponge. The backing may permit the sponge to be packaged, handled, and/or applied to a wound in a sterile and secure manner. The backing may be made of cloth, plastic, paper, film, and/or any suitable material. The backing may be attached to at least one surface of the sponge with an adhesive, stitching, staples, and/or any suitable fastener.

In another embodiment, the disclosure also provides a method of making a chitosan based wound dressing comprising dissolving the components in a solvent to form a solution, and freeze drying the solution to form a hemostatic sponge.

In one embodiment, the method of making a chitosan based sponge includes providing a dry powder formulation comprising the individual components; mixing with purified water in an amount of about 3.5 to about 4% by weight to form a mixture; and lyophilizing (freeze drying) the mixture; then followed by humidification and compression, packaging and final sterilization. In one embodiment, the dry powder is mixed with purified water in an amount of about 3.75% by weight; the mixture is subsequently lyophilized; then followed by humidification and compression, packaging and final sterilization.

In some more specific embodiments, the method of making a chitosan sponge comprises: a) weighing and adding each component to the stirred distilled water to form a mixture, and stirring the mixture to a complete homogeneity; b) transferring the homogeneous mixture into an ultra-low freezer for a complete freezing; c) transferring the frozen mixture to a freeze dryer for freeze drying until complete drying; and d) removing the dried material from the freeze dryer (the material should be at its driest state and can be cut by razor blade to size). In some embodiments, the method of making a chitosan sponge further or optionally includes: allowing the dried material to pick up moisture from the air until it equilibrates; compressing the moistened material (sponge) using a flat surface such as pyrex glass or PTFE sheets; and sealing and sterilizing the sponge into different shapes and sizes.

In some embodiments, the method of making the wound dressings of the present disclosure includes the steps comprising dissolving the appropriate ingredients to be lyophilized in an appropriate solvent to prepare a homogenous solution. The homogenous solution is subjected to a freezing and vacuum drying cycle. The freezing/drying step phase removes the solvent by sublimation, leaving a porous structure. The lyophilization conditions are important to the novel porous structure in order to create a large matrix surface area in the hemostat with which body fluids can interact once the dressing is applied to a wound requiring hemostasis.

During the lyophilization process, several parameters and procedures are important to produce wound dressings having mechanical properties suitable for use in hemostatic wound dressings. The features of such microporous structure can be controlled to suit a desired application by choosing the appropriate conditions to form the composite hemostat during lyophilization. The type of microporous morphology developed during the lyophilization is a function of such factors as the solution thermodynamics, freezing rate, temperature to which it is frozen, and concentration of the solution. To maximize the surface area of the porous sponge of the present disclosure, a preferred method is to quickly freeze the homogeneous solvent at lower than 0° C., preferably at about −50° C., and to remove the solvent under high vacuum. The porous sponge produced thereby provides a large fluid-absorbing capacity to the hemostatic wound dressing. When the hemostatic wound dressing comes into contact with body fluid, a very large surface area of the sponge is exposed to the fluid instantly. The hydration force of the hemostat and subsequent formation of a tacky gelatinous layer helps to create an adhesive interaction between the hemostatic sponge and the bleeding site. The formation of a gelatinous sheet on oxidized cellulose upon blood contact will enhance the sealing property of the water-soluble gelatinous layer, which is critical to rapid hemostasis in cases ranging from moderate to severe bleeding.

In one embodiment, the disclosure provides a method of treating a wound/surgery/bleeding comprising administering a chitosan based hemostatic sponge to a wound/surgery/bleeding site of a subject. In an embodiment, the surgery is a nasal surgery.

In certain embodiments, the chitosan based hemostatic sponges are effective in providing and maintaining hemostasis in cases of severe bleeding. As used herein, severe bleeding is meant to include those cases of bleeding where a relatively high volume of blood is lost at a relatively high rate. Examples of severe bleeding include, without limitation, bleeding due to arterial puncture, liver resection, blunt liver trauma, blunt spleen trauma, aortic aneurysm, bleeding from patients with over-anticoagulation, or bleeding from patients with coagulopathies, such as hemophilia. Such wound dressings allow a patient to ambulate quicker than the current standard of care following, e.g. a diagnostic or interventional endovascular procedure.

The hemostatic sponge of the present disclosure provides and maintains effective hemostasis when applied to a wound/surgery/bleeding requiring hemostasis. Effective hemostasis, as used herein, is the ability to control and/or abate capillary, venous, or arteriole bleeding within an effective time, as recognized by those skilled in the art of hemostasis. Further indications of effective hemostasis may be provided by governmental regulatory standards and the like. In one embodiment, the disclosure also provides use of a chitosan based sponge in treating a wound/surgery/bleeding condition of a subject. In an embodiment, the use of a chitosan based sponge is for nasal surgery. In an embodiment, the use of a chitosan based sponge is for nasal packing.

In certain embodiments, the present disclosure provides a kit for effecting hemostasis at a site of wound/surgery/bleeding of a subject, the kit comprising: a sponge comprising about 45% to about 95% by weight carboxymethyl chitosan and about 4% to about 12% by weight methyl cellulose, and instructions for applying the sponge to the site of wound/surgery/bleeding. In an embodiment, the hemostasis is for nasal surgery. In an embodiment, the chitosan sponge is used for nasal packing.

In certain embodiments, the present disclosure provides a kit for effecting hemostasis at a site of wound/surgery/bleeding of a subject, the kit comprising: a sponge comprising about 45% to about 95% by weight carboxymethyl chitosan, about 4% to about 12% by weight methyl cellulose, about 5% to about 15% by weight hydroxy ethyl cellulose, and about 10% or less by weight calcium alginate, and instructions for applying the sponge to the site of wound/surgery/bleeding. In an embodiment, the hemostasis is for nasal surgery. In an embodiment, the chitosan sponge is used for nasal packing.

In certain embodiments, the present disclosure provides a kit for effecting hemostasis at a site of wound/surgery/bleeding of a subject, the kit comprising: a sponge comprising about 70% to about 80% by weight carboxymethyl chitosan, about 5% to about 10% by weight methyl cellulose, about 10% to about 15% by weight hydroxy ethyl cellulose, and about 2% to about 6% by weight calcium alginate, and instructions for applying the sponge to the site of wound/surgery/bleeding. In an embodiment, the hemostasis is for nasal surgery. In an embodiment, the chitosan sponge is used for nasal packing.

In certain embodiments, the present disclosure provides a kit for effecting hemostasis at a site of wound/surgery/bleeding of a subject, the kit comprising: a sponge consisting essentially of about 45% to about 95% by weight carboxymethyl chitosan, about 4% to about 12% by weight methyl cellulose, about 5% to about 15% by weight hydroxy ethyl cellulose, and about 10% or less by weight calcium alginate, and instructions for applying the sponge to the site of wound/surgery/bleeding. In an embodiment, the hemostasis is for nasal surgery. In an embodiment, the chitosan sponge is used for nasal packing.

In certain embodiments, the present disclosure provides a kit for effecting hemostasis at a site of wound/surgery/bleeding of a subject, the kit comprising: a sponge consisting essentially of about 45% to about 95% by weight carboxymethyl chitosan, about 4% to about 12% by weight methyl cellulose, about 5% to about 15% by weight hydroxy ethyl cellulose, about 10% or less by weight calcium alginate, and about 10% or less by weight sodium acrylate, and instructions for applying the sponge to the site of wound/surgery/bleeding. In an embodiment, the hemostasis is for nasal surgery. In an embodiment, the chitosan sponge is used for nasal packing.

In certain embodiments, the present disclosure provides a kit for effecting hemostasis at a site of wound/surgery/bleeding of a subject, the kit comprising: a sponge consisting essentially of about 70% to about 80% by weight carboxymethyl chitosan, about 5% to about 10% by weight methyl cellulose, about 10% to about 15% by weight hydroxy ethyl cellulose, and about 2% to about 6% by weight calcium alginate, and instructions for applying the sponge to the site of wound/surgery/bleeding. In an embodiment, the hemostasis is for nasal surgery. In an embodiment, the chitosan sponge is used for nasal packing.

In certain embodiments, the present disclosure provides a kit for effecting hemostasis at a site of wound/surgery/bleeding of a subject, the kit comprising: a sponge consisting essentially of about 70% to about 80% by weight carboxymethyl chitosan, about 5% to about 10% by weight methyl cellulose, about 10% to about 15% by weight hydroxy ethyl cellulose, about 2% to about 6% by weight calcium alginate, and about 5% or less by weight sodium acrylate, and instructions for applying the sponge to the site of wound/surgery/bleeding. In an embodiment, the hemostasis is for nasal surgery. In an embodiment, the chitosan sponge is used for nasal packing.

In certain embodiments, the present disclosure provides a kit for effecting hemostasis at a site of wound/surgery/bleeding of a subject, the kit comprising: a sponge consisting essentially of about 76% by weight carboxymethyl chitosan, about 10% by weight methyl cellulose, about 12% by weight hydroxy ethyl cellulose, and about 5% by weight calcium alginate, and instructions for applying the sponge to the site of wound/surgery/bleeding. In an embodiment, the hemostasis is for nasal surgery. In an embodiment, the chitosan sponge is used for nasal packing.

### EXAMPLES

### Example 1: Preparation of Chitosan Sponges

Porous chitosan sponges were made through the process of lyophilization (freeze drying). Cellulose derivatives were added to the carboxymethyl chitosan mixture to enhance or change the properties of the resultant sponge including extending the degradation time and increasing the softness (pliability) of the sponge. The derivatives used in the test included methyl cellulose (CMC), and hydroxy ethyl cellulose (HEC). In addition, calcium alginate was also used to improve some of the desired properties. Comparisons were made for the hydration and degradation behavior of several porous chitosan sponge prototypes against existing chitosan pack Posisep®, which was distributed by Hemostasis.

### Materials

| | Carboxymethyl | Methyl | Hydroxyethyl | Calcium |
|---|---|---|---|---|
| Prototype | Chitosan¹ | Cellulose² | Cellulose (HEC)³ | Alginate⁴ |
| MCC1 | 75% | 12.5 | 12.5% | 0% |
| MCC2 | 77% | 8% | 11% | 4% |
| MCC3 | 80% | 8% | 12% | 0% |
| MCC4 | 77% | 8% | 11% | 4% |
| MCC5 | 80.5% | 7% | 12.5% | 0% |
| MCC6 | 75% | 7% | 12% | 6% |
| MCC7 | 76% | 7% | 12% | 5% |
| MCC8 | 77% | 7% | 13% | 5% |
| MCC9 | 79% | 6% | 9% | 6% |
| MCSP2TCIMC | 75% | 7% (from TCI | 13% (from Spectrum | 5% |
| | | (7,000-10,000 mPa*s) | 3,400 cps) | |
| MCMP4KSp2PB_3 | 76% | 7% (from MP | 12% (From Spectrum | 5% (from Pfaltz |
| | | biomedical (4000 cps) | 3,400 cps) | and Bauer) |
| MCMP4KSp2PB_4 | 80% | 6% (from MP | 10% (from Spectrum | 4% (from Pfaltz |
| | | biomedical (4000 cps)) | 3,400 cps) | and Bauer) |
| MC(Control)Th | 78% | 7% | 10% | 5% |

¹Carboxymethyl chitosan all from Heppe Medical.
²Methyl Cellulose is from Alfa Aesar (8000 cps) unless otherwise indicated.
³Hydroxyethyl Cellulose is from Amresco (high purity grade, pH 6.0) unless otherwise indicated.
⁴Calcium Alginate is from TCL unless otherwise indicated.

### Procedure for Chitosan Sponge Fabrication:

1. Uniquely labeling all test molds, assembling test setup, recording all equipment used, determining total volume of sponge to make, and adding distilled water to stainless steel mold with stir bar, usually 12 mL;
2. Multiplying the volume by the dry weight % to give the total weight of solids in the mixture, multiplying the total weight of solids by the mass % of the component to get the weight of each component, and weighing and adding each component to the stirred distilled water;
3. When solutions became thick, or perhaps too viscous to stir, gentle heat was applied, but not more than 30° C., and all components were stirred until the hydrogel mixture was completely homogenous;
4. Removing the stir bar, and transferring into the ultra-low freezer. It took about 15-20 minutes to ensure complete freezing;
5. Starting the manual cooling of the freeze dryer until the coil was down to temperature (around −54° C.), and transferring the samples to the freeze dryer, sealing all ports and placing the cover over the top, then engaging the automatic mode until the coil was down to the correct temperature;
6. Waiting for about 24 hours for complete drying, this varied depending on the amount of water to be removed and the polymer wt % used in the sponge. A dried sponge was obtained;
7. Removing samples from the freeze dryer. They were at their driest state and could be cut by razor blade to size, and samples slowly picked up moisture from the air, unless vacuum sealed, until they equilibrated usually at about 5-12% water by weight. This process usually took 24-48 hours at ambient conditions. Once moisturized, sponges were more pliable and compressible, but more difficult to cut with a razor blade;
8. A pliable sponge was compressed using a flat surface, which could be pyrex glass or PTFE sheets. Once hydrated, the sponge would expand preferentially in the direction it was previously compressed.

### Example 2: Hydration Test of the Chitosan Sponges

### Procedure for Chitosan Sponge Hydration:

1. Appropriate sized specimens (usually 0.08 g to 0.15 g) were weighed in the dry state and results were recorded;
2. The scale was tared to include the weight of the sponge itself;
3. Saline was added to the sponge in small (0.25 mL) increments.
4. After complete hydration, any extra saline (collecting/pooling outside of the sponge) was blotted with a dry paper towel.
5. Specimen was weighed again, and the weight of the added saline was recorded.
6. Hydration was determined by (weight of saline/weight of dry sponge). The units were expressed as mL saline/g sponge, or mL/g.

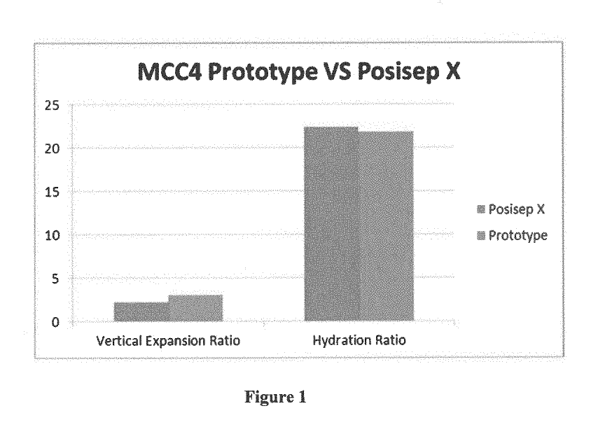

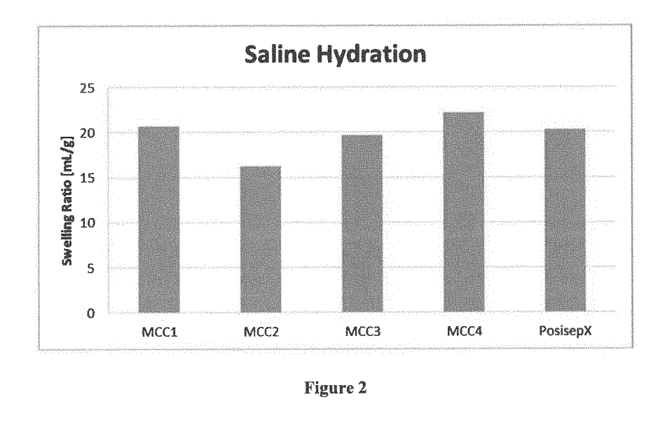

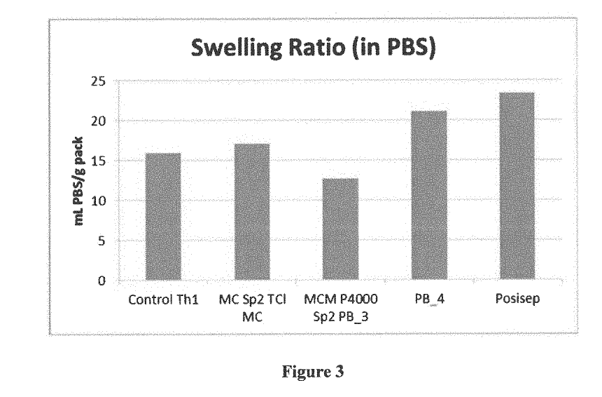

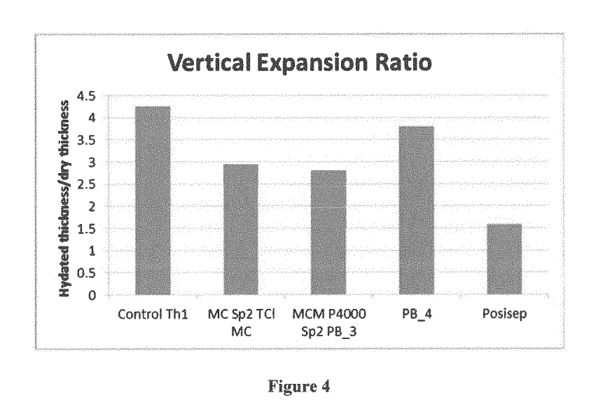

FIG. 1 shows a comparison chart of hydration ratio and vertical expansion ratio of one prototype against PosiSep® X, also distributed by Hemostasis. The chart shows that the prototype product possessed similar hydration ratio and vertical expansion ratio properties as Posisep® X. FIG. 2 shows that additional prototypes were about equivalent to or better than PosiSep® X product in terms of saline hydration ratio. FIG. 3 shows that additional prototypes were about equivalent to PosiSep® X product in terms of swelling ratio. The swelling ratio is a measure of how much fluid a solid can absorb before becoming saturated. FIG. 4 shows the vertical expansion properties of the prototypes and Posisep®. All the prototypes exceeded the Posisep® product in terms of vertical expansion ratio. This vertical expansion, together with the turgidity (firmness), degradation timeframe, hydration speed and handling (tackiness) of the pack, contributes to its ultimate performance.

### Example 3: Degradation Test of the Chitosan Sponges

### Procedure for Chitosan Sponge Degradation:

1. After the hydration test, specimens continued on to the degradation test;
2. Specimens were added to a pyrex bottle with 40 mL PBS;
3. The pyrex bottle were added to the heated shaker bath, the temperature was set to 37° C. and the speed was set to 30 RPM;
4. After each day, the samples were observed and a qualitative determination of degradation was graded between 0 (no degradation) and 10 (complete degradation);
5. On the 8th day, the samples were removed from PBS and rinsed in deionized water.
6. The leftover sponge material was added to a pre-weighed piece of filter paper and left to dry for several days;
7. Final drying occurred in the environmental chamber at 55° C. for 20-30 minutes;
8. Final dry weight was measured and recorded; and
9. The percentage of degradation was determined by 1-(final dry weight/original dry weight).

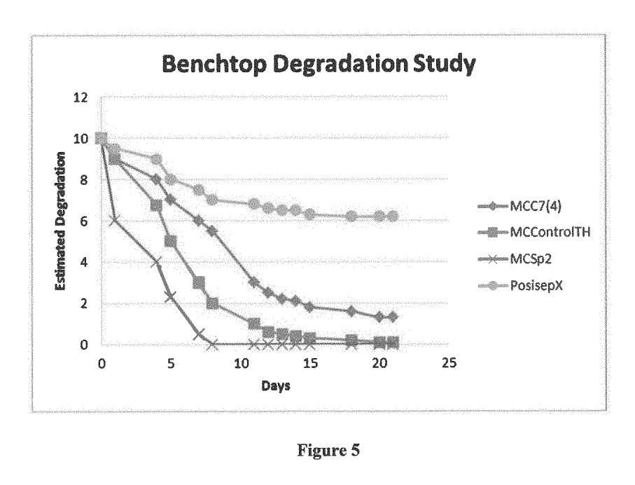

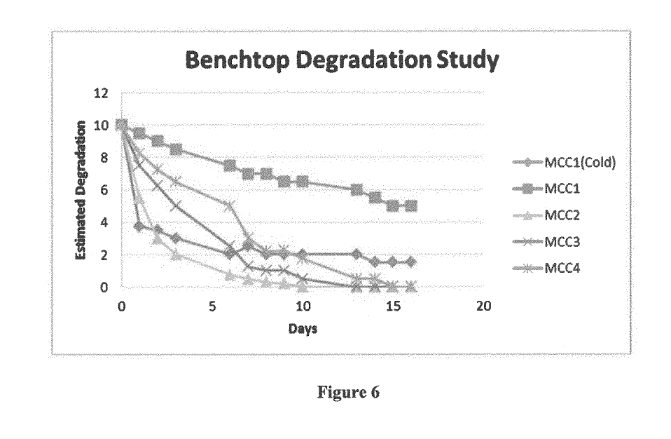

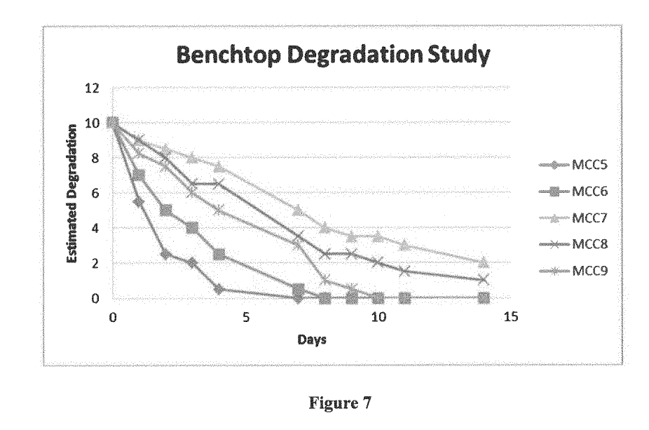

It's believed that a quick and uniform degradation that is complete within 14 days is desirable. FIGS. 5 to 7 show that the prototypes of this disclosure had quicker degradation (while maintaining early firmness), and had more “suctionable” remnants than PosiSep® X.

### Example 4: Pliability Measurements of the Chitosan Sponges

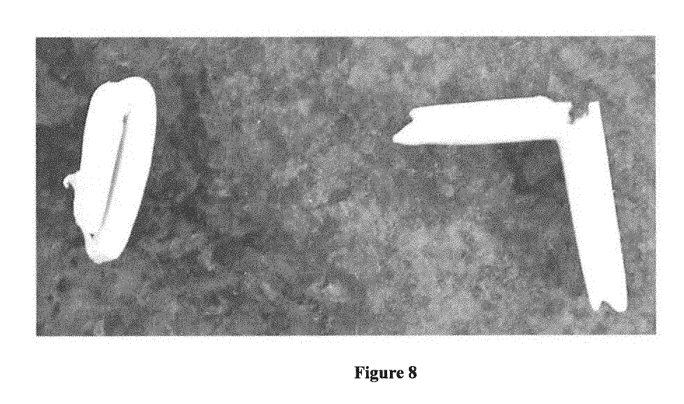

Pliability comparisons were made between prototype (MCC9) and PosiSep® X. Attempts were made to form the packing material around different curvature materials such as a light bulb or a stainless steel dish. It was found that the prototype sponge was able to bend and fold (fold in half without breaking), while PosiSep® X wafer broke when it was flexed (FIG. 8: prototype MCC9 on the left, PosiSep® X on the right). FIG. 9 shows how to measure a bending angle of a sponge further in support of the great pliability of the chitosan sponges as disclosed herein. The bending angle was measured as the angle formed when one part of a sponge was fixed and the other part was bent or rotated until the sponge cracked or broke.

In conclusion, these studies have shown that these prototypes have been able to exceed or match the hydration and degradation performance of PosiSep® or PosiSep® X. Further studies will focus on identifying additional properties of the chitosan material such as molecular weight on the porosity and pore size and on identifying additional process parameters to make even better chitosan based hemostatic sponges.

### INCORPORATION BY REFERENCE

The contents of all references, patents, pending patent applications and published patents, cited throughout this application are hereby expressly incorporated by reference.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed. It is intended that the following claims define the scope of the disclosure and that methods and structures within the scope of these claims and their equivalents be covered thereby.

1. A hemostatic formulation comprising carboxymethyl chitosan in an amount of about 45% to about 95% by weight, and methyl cellulose in an amount of about 4% to about 12% by weight.

2. The hemostatic formulation of claim 1, wherein the formulation further comprises hydroxy ethyl cellulose in an amount of about 5% to about 15% by weight.

3. The hemostatic formulation of claim 1, wherein the formulation further comprises calcium alginate in an amount of about 10% or less by weight.

4. The hemostatic formulation of claim 1, wherein the formulation further comprises sodium polyacrylate in an amount of about 10% or less by weight.

5-6. (canceled)

7. The hemostatic formulation of claim 1, wherein the formulation comprises carboxymethyl chitosan in an amount of about 70% to about 80% by weight, and methyl cellulose in an amount of about 5% to about 10% by weight.

8. The hemostatic formulation of claim 1, wherein the formulation comprises carboxymethyl chitosan in an amount of about 45% to about 95% by weight, methyl cellulose in an amount of about 4% to about 12% by weight, hydroxy ethyl cellulose in an amount of about 5% to about 15% by weight, and calcium alginate in an amount of about 10% or less by weight.

9. The hemostatic formulation of claim 1, wherein the formulation comprises carboxymethyl chitosan in an amount of about 70% to about 80% by weight, methyl cellulose in an amount of about 5% to about 10% by weight, hydroxy ethyl cellulose in an amount of about 5% to about 15% by weight, and calcium alginate in an amount of about 10% or less by weight.

10-17. (canceled)

18. The hemostatic formulation of claim 1, wherein the formulation further comprises a binding agent, a clotting accelerator, or one or more therapeutic agents.

19. A hemostatic sponge comprising carboxymethyl chitosan in an amount of about 45% to about 95% by weight, and methyl cellulose in an amount of about 4% to about 12% by weight.

20. The hemostatic sponge of claim 19, wherein the sponge further comprises hydroxy ethyl cellulose in an amount of about 5% to about 15% by weight.

21. The hemostatic sponge of claim 19, wherein the sponge further comprises calcium alginate in an amount of about 10% or less by weight.

22. The hemostatic sponge of claim 19, wherein the sponge further comprises sodium polyacrylate in an amount of about 10% or less by weight.

23-26. (canceled)

27. The hemostatic sponge of claim 19, wherein the sponge comprises carboxymethyl chitosan in an amount of about 70% to about 80% by weight, methyl cellulose in an amount of about 5% to about 10% by weight, hydroxy ethyl cellulose in an amount of about 5% to about 15% by weight, and calcium alginate in an amount of about 10% or less by weight.

28. The hemostatic sponge of claim 19, wherein it consists essentially of carboxymethyl chitosan in an amount of about 45% to about 95% by weight, methyl cellulose in an amount of about 4% to about 12% by weight, hydroxy ethyl cellulose in an amount of about 5% to about 15% by weight, and calcium alginate in an amount of about 10% or less by weight.

29-34. (canceled)

35. The hemostatic sponge of claim 19, wherein it consists essentially of carboxymethyl chitosan in an amount of about 45% to about 95% by weight, methyl cellulose in an amount of about 4% to about 12% by weight, hydroxy ethyl cellulose in an amount of about 5% to about 15% by weight, calcium alginate in an amount of about 10% or less by weight, and sodium polyacrylate in an amount of about 10% or less by weight.

36-37. (canceled)

38. The hemostatic sponge of claim 19, wherein the sponge has a vertical expansion ratio of 2 or more.

39. The hemostatic sponge of claim 19, wherein the sponge has a bench degradation rate of less than 20 days.

40. (canceled)

41. The hemostatic sponge of claim 19, wherein the sponge has a pliability of over 70 degree.

42. A method of making a hemostatic sponge according to claim 19, the method comprising the steps of: providing individual components of a hemostatic formulation; dissolving the individual components in a solvent to form a solution; and freezing drying the solution to form a hemostatic sponge.

43. The method of claim 42, wherein the solvent is purified water.

44. (canceled)
